(19) United States
(12) Patent Application Publication
(10) Pub. No.: US 2009/0325862 A1
(43) Pub. Date: Dec. 31, 2009

(54) Histone Deacetylase Inhibitors for the Treatment of Neurodegeneration

(76) Inventors: Christian Steinkuhler, Rome (IT); Gretchen Bain, San Diego, CA (US); John Trauger, San Diego, CA (US)

Correspondence Address: MERCK AND CO., INC, P O BOX 2000, RAHWAY, NJ, 07065-0907, US

(21) Appl. No.: 12/227,055

(22) PCT Filed: Apr. 30, 2007
(86) PCT No.: PCT/US2007/010563

§ 371 Date: May 4, 2009

Related U.S. Application Data
(60) Provisional application No. 60/797,621, filed on May 4, 2006.
(60) Provisional application No. 60/832,915, filed on Jul. 24, 2006.

(51) Int. Cl. A61K 38/12; A61K 31/44; A61K 31/19; G01N 33/53

(52) U.S. Cl. 514/9; 514/357; 514/557; 435/7.1

(57) ABSTRACT

The instant invention is directed to methods for treating neurodegenerative diseases comprises administering an effective amount of a selective histone deacetylase 8 inhibitor to a patient in need thereof.

### FIELD OF THE INVENTION

The invention is directed to the use of histone deacetylase inhibitors for the treatment of neurodegeneration. In particular, the invention is directed to methods of treating patients for neurodegenerative diseases by administering to the patient a selective histone deacetylase 8 inhibiting compound. The invention is also directed to pharmaceutical compositions for treatment of neurodegenerative diseases, and methods of manufacturing pharmaceutical compositions.

### BACKGROUND OF THE INVENTION

Chromosomes are complexes formed from DNA and proteins found in multicellular organisms. One type of chromosomes, called nucleosomes, are an important element of gene expression. Histone proteins are the major structural proteins found in nucleosomes, and the acetylation of histone proteins alters the biological properties of chromosomes. Histone deacetylases (HDACs) are a family of enzymes that inhibit the acetylation of histones, and can thereby play a key role in the regulation of gene expression and DNA repair.

Gene expression is often impacted by tumors, and inhibitors of HDACs have been studied in tumors. Some HDAC inhibitors have shown antitumor activity in preclinical models and in clinical trials. See, e.g. Marks et al, Clin Canc Res 2001, 7:759-760.

It has also been postulated that HDACs play a role in neurodegeneration. See, e.g., Langley et al, Curr Drug Targets CNS Neurol Disord. 2005 February; 4(1):41-50. For example, the HDAC inhibitor suberoylanilide hydroxamic acid (SAHA) has been shown to cross the blood-brain barrier, and cause accumulation of acetylated histones in the brain. See WO2003083067. Hockly et al, PNAS 2003, 100(4), 2041-2046, suggests that HDAC inhibitors may be generally useful in treating neurodegenerative diseases such as Huntington's Disease.

There are at least 18 known subtypes of the HDAC enzymes. The HDAC subtypes have been divided into three classes. See Gregoretti et al, J. Mol. Biol., 2004, 338, 17-31). Class I HDACs, which include HDAC subtypes 1-3 and 8, are homologous to the yeast Rpd3 deacetylase. Class II HDACs, which include HDAC subtypes 4-7, 9, and 10, are related to the yeast Hda1 deacetylase. Class I HDACs are thought to be expressed in most cell types, while Class II HDACs have a more restricted expression pattern. See Annemieke et al, Biochem J2003, 370:737-749. Class III HDACs, which are also known as the SIR2 family of proteins, have homology to both class I and II enzymes, but cannot unambiguously be assigned to either class. HDAC 11, which has only recently been identified, has not yet been classified in one of the HDAC classes. See Annemieke.

Class I and II HDACs, as well as HDAC11, are zinc-dependent hydrolases. According to Johnstone, Nat Rev Drug Discovery, 2002, 1, 287-298, the therapeutically relevant HDAC inhibitors are thought to be nonselective or poorly selective inhibitors of all or most of class I and II enzymes, but not to inhibit class III HDACs. With respect to the antitumor properties of HDAC inhibitors, it is not know whether lack of specificity contributes to the efficacy, or whether the antitumor properties correlate with specific HDAC subtypes.

HDAC8 is a 377 amino acid residue enzyme which maps to the X chromosome. See Somoza et at, Structure 2004, 12, 1325-1334. Vannini et al, PNAS 2004, 101, 15064-1506, identified the crystal structure of HDAC subtype 8 in complexation to the hydroxamic acid inhibitor N-hydroxy-4-{methyl[(5-pyridin-2-yl-2-thienyl)sulfonyl]amino}benzamide. Vannini et al demonstrated that HDAC8 is important for the growth of human tumor cell lines and has a distinct inhibition pattern that differs from that of HDAC1 and HDAC3, which both share 43% sequence identity with HDAC8.

Applicants have now discovered that HDAC inhibitors having a specific binding and HDAC inhibition pattern, including selective affinity for the HDAC8 subtype, are of particular efficacy in the treatment of neurodegeneration.

### SUMMARY OF THE INVENTION

The invention is directed to methods of treating neurodegenerative diseases, comprising administering a therapeutically effective amount of a selective HDAC8 inhibiting compound to a patient in need thereof. The invention is also directed to the use of a selective HDAC8 inhibitor of the invention for the manufacture of a medicament for treating neurodegenerative diseases, comprising combining a selective HDAC8 inhibitor of the invention with a pharmaceutically acceptable carrier or diluent.

### DETAILED DESCRIPTION OF THE INVENTION

The invention is directed to methods of treating neurodegenerative diseases, comprising administering a therapeutically effective amount of a selective HDAC8 inhibiting compound to a patient in need thereof, wherein the compound is not (2R)-2-proplyoctanoic acid (arundic acid).

In one embodiment, the selective HDAC8 inhibitors for use in the invention are selective for the HDAC8 subtype over all other HDAC subtype receptors. In another embodiment, the selective HDAC8 inhibitors for use in the invention are selective for the HDAC1, HDAC3 and HDAC8 subtypes over all other HDAC subtype receptors. Preferably, the selective HDAC8 inhibitor is selective for each of the HDAC1, HDAC3 and HCDAC8 subtypes within a factor of five. Most preferably, the selective HDAC8 inhibitor is selective for each of the HDAC1, HDAC3 and HCDAC8 within a factor of five, and the selective HDAC8 inhibitor is more selective for the HDAC8 subtype than for the HDAC1 and HDAC3 subtype.

In one preferred embodiment, the selective HDAC8 inhibitors for use in the invention are selective for each of the HDAC1, HDAC3 and HCDAC8 subtypes within a factor of five. Thus if the selective HDAC8 inhibitor of the invention has an IC₅₀ of 100 μM with respect to HDAC8, the inhibitor has an IC₅₀ of less than 500 μM with respect to HDAC1 and HDAC3. Alternatively, if the selective HDAC8 inhibitor of the invention has an IC₅₀ of 100 μM with respect to HDAC1, the inhibitor has an IC₅₀ of less than 500 μM with respect to HDAC3 and HDAC8. In another embodiment, if the selective HDAC8 inhibitor of the invention has an IC₅₀ of 100 μM with respect to HDAC3, the inhibitor has an IC₅₀ of less than 500 μM with respect to HDAC1 and HDAC8. In preferred aspect of this embodiment, the compound of the invention is most selective for HDAC8.

In this embodiment, if a compound has an inhibitory value for HDAC8 of a value n, then the compound has a counterpart inhibitory value for each of HDAC3 and HDAC8 of no more than 5n.

Exemplary binding patterns for hypothetical selective HDAC8 inhibitor compounds A-I of the invention are shown below in Table 1:

TABLE 1
HDAC Binding Properties of Selective HDAC8 Inhibitors

| Hypothetical | | | | | | |
|---|---|---|---|---|---|---|
| Compound | HDAC8 IC₅₀ | | HDAC1 IC₅₀ | | HDAC3 IC₅₀ | |
| A | 100 | μM | Up to 500 | μM | up to 500 | μM |
| B | 50 | μM | Up to 250 | μM | up to 250 | μM |
| C | 25 | μM | Up to 125 | μM | up to 125 | μM |
| D | 10 | μM | Up to 50 | μM | up to 50 | μM |
| E | 1000 | nM | Up to 5000 | nM | up to 5000 | nM |
| F | 500 | nM | Up to 2500 | nM | up to 2500 | nM |
| G | 250 | nM | Up to 1250 | nM | up to 1250 | nM |
| H | 100 | nM | Up to 500 | nM | up to 500 | nM |
| I | 50 | nM | Up to 250 | nM | up to 250 | nM |

In another aspect, the invention is directed to a method of identifying a compound useful for treatment of neurodegenerative diseases from a group of compounds, comprising assaying the binding affinity of a group of compounds for each of the HDAC1, HDAC3 and HDAC8 subtypes, determining the IC₅₀ value for each of the compounds in the group for each of the HDAC1, HDAC3 and HDAC8 subtypes, and selecting those compounds from the group in which the IC₅₀ value for each of the HDAC1, HDAC3 and HDAC8 subtypes is within a factor of five. In distinct aspects of this embodiment, the compounds to be selected have an IC₅₀ value for the HDAC8 subtype of less than one of the following threshold values: 100 μM, 50 μM, 25 μM, 10 μM, 1000 nM, 500 nM, 250 nM, 100 nM or 50 nM. Suitable assays for determining HDAC1, HDAC3 and HDAC8 binding affinities are known to those skilled in the art, and include the assays described herein.

In still another embodiment, the invention is directed to the use of a selective HDAC8 inhibitor of the invention for the manufacture of a medicament for treating neurodegenerative diseases, comprising combining a selective HDAC8 inhibitor of the invention with a pharmaceutically acceptable carrier or diluent, wherein the selective HDAC8 inhibitor is not (2R)-2-propyloctanoic acid.

In another embodiment, the invention is directed to a pharmaceutical composition for treatment of neurodegenerative diseases, comprising a selective HDAC8 inhibitor of the invention, wherein the selective HDAC8 inhibitor is not (2R)-2-propyloctanoic acid. Preferably, the selective HDAC8 inhibitors for use in this embodiment are selective for each of the HDAC1, HDAC3 and HCDAC8 subtypes within a factor of five. Most preferably for use in this embodiment, the selective HDAC8 inhibitor is selective for each of the HDAC1, HDAC3 and HCDAC8 within a factor of five, and the selective HDAC8 inhibitor is more selective for the HDAC8 subtype than for the HDAC1 and HDAC3 subtype.

As used herein, the term “HDAC8” refers to subtype 8 of the HDAC enzyme family, as known by those of ordinary skill in the art. The HDAC family is generally described in Annemieke et al, Biochem J2003, 370:737-749. HDAC8 is further described in Somoza et al, Structure 2004, 12, 1325-1334 and Vannini et al, PNAS2004, 101, 15064-1506.

A “selective HDAC8 inhibitor” refers to a compound which is an inhibitor of the HDAC8 subtype. In general, HDAC8 inhibitors can be identified as those compounds which when assayed in the HDAC8 assay described herein, have an IC₅₀ of less than or equal to 100 μM, more preferably less than 50 μM, more preferably less than 25 μM, and even more preferably less than 10 μM. Other, more selective preferred HDAC 8 inhibitors, when assayed in the HDAC8 assay described herein, have an IC₅₀ of less than or equal to 1000 nM, more preferably less than or equal to 500 nM, more probably less than or equal to 250 mM, even more preferably less than or equal to 100 nM.

As used herein, the term “binding affinity” is a measure of the physicochemical interaction between a radiolabelled ligand and its specific receptor in vitro. One measure of binding affinity is the inhibitory concentration or IC₅₀ value, which is the concentration of unlabeled radioligand (or ligand of interest, for example a selective HDAC8 inhibitor of the type contemplated for use in this invention) which is required to inhibit 50% of the specific binding of the radiolabelled radioligand. The IC₅₀ value can be determined by various competitive binding assays known to those skilled in the art.

The compounds of the present invention have utility in treating, ameliorating or controlling neurodegenerative diseases or disorders. As used herein, the term “neurodegeneration” or “neurodegenerative diseases or disorders” refer to diseases or disorders that are characterized by the degeneration of nervous system tissue, and include diseases that involve a wide range of pathologies. Exemplary neurodegenerative diseases and disorders include disorders characterized by progressive dementia, including dementia (including vascular dementia, pre-senile and senile dementia); Alzheimer's Disease; multiple sclerosis; amyotrophic lateral sclerosis (ALS); Creutzfeldt-Jakob Disease; prion-related diseases; stroke, traumatic brain injury and spinal cord injury; Pick's disease; Huntington's Disease; multiple system atrophy including dementia; progressive supranuclear palsy; Lewy body disease; corticobasal degeneration; syndromes including abnormalities of posture and movement, including Parkinson's Disease, Tourette's Syndrome, familial tremor, torsion dystonia; syndromes of progressive ataxia, including cerebellar degenerations, spinocerebellar degeneration; syndromes of muscular weakness and wasting, including Charcot-Marie-Tooth disease, chronic progressive neuropathies; and syndromes of progressive visual loss, including retinitis pignientosa and hereditary optic atrophy.

The subject or patient to whom the compounds of the present invention is administered is generally a human being, male or female, in whom treatment of a neurodegenerative disease or disorder is desired, but may also encompass other mammals, such as dogs, cats, mice, rats, cattle, horses, sheep, rabbits, monkeys, chimpanzees or other apes or primates, for which treatment of a neurodegenerative disease or disorder is desired.

As used herein, the term “treatment” or “treating” means any administration of a compound of the present invention and includes (1) inhibiting a neurodegenerative disease or the symptoms of a neurodegenerative disease in an animal that is experiencing or displaying the pathology or symptomatology of a neurodegernative disease (i.e., arresting further development of the pathology and/or symptomatology, such as by enhancing plasticity), or (2) ameliorating a neurodegenerative disease or the symptoms of a neurodegenerative disease in an animal that is experiencing or displaying the pathology or symptomatology of a neurodegenerative disease (i.e., reversing the pathology and/or symptomatology). The term “controlling” includes preventing, treating, eradicating, ameliorating or otherwise reducing the severity of a neurodegenerative disease.

In one embodiment, the neurodegenerative disease or disorder for which the selective HDAC8 inhibitors are useful is stroke, and the neurological injuries caused by stroke. As used herein, the term “stroke” refers to a clinical event involving impairment of cerebral circulation, resulting in neurological injury. Typically, stroke is manifest by the abrupt onset of a focal neurological deficit. Stroke results from a rupture or obstruction (as by a thrombus or embolus) of an artery of the brain.

As used herein, the term “ischemic stroke” refers to stroke characterized by localized tissue anemia due to obstruction of the inflow of arterial blood. Ischemic stroke is usually caused by atherothrombosis or embolism of a major cerebral artery, but may also be caused by coagulation disorders or nonatheromatous vascular disease.

A second type of stroke, hemorrhagic stroke, involves a hemorrhage or rupture of an artery leading to the brain. Hemorrhagic stroke results in bleeding into brain tissue, including the epidural, subdural, or subarachnoid space of the brain. A hemorrhagic stroke typically results from the rupture of an arteriosclerotic vessel that has been exposed to arterial hypertension or to thrombosis.

During acute ischemic stroke, i.e., the period from the cerebrovascular event up to 24 hours after the event, the arterial occlusion results in an immediate infarcted core of brain tissue, where cerebral blood flow is significantly reduced, for example to less than 20% of the normal blood flow. The infarcted core suffers irreversible damage due to significant cell death.

One class of stroke patients to which the selective HDAC8 inhibitor of the invention may be administered is a patient at risk for stroke. As used herein, the term “patient at risk for stroke” means an individual who has had a previous stroke, or has a risk factor for stroke. Known risk factors for stroke include atherosclerosis, arterial hypertension, lipohyalinosis, hyperlipidemia, hypercholesterolemia, atrial fibrillation, smoking, inflammatory markers (including C-reactive protein), infection, homocysteine, sleep-disordered breathing, cerebral autosomal dominant arteriopathy with subcortial infarcts and leuko-encephalopathy (CADASIL), migraine headaches, sickle-cell anemia, antiphospholipid antibody syndrome, arterial dissection, cocaine abuse and obesity.

A second class of stroke patients to which a compound of the invention may be administered are acute stroke patients, i.e., patients who have suffered ischemic stroke within the last 7 days. One preferred class of acute stroke patients are those who have suffered stroke within the last 3 days. A more preferred class of acute stroke patients are those who have suffered stroke within the last 48 hours, even more preferably within the last 24 hours. As common in the art of treating stroke, patients may be classified according to the period of time when stroke occurred. So, for example, one class of acute stroke patients are those who have suffered stroke within the last 18 hours. Another class of acute stroke patients are those who have suffered stroke within the last 12 hours. Another class of acute stroke patients are those who have suffered stroke within the last 8 hours. Another class of acute stroke patients are those who have suffered stroke within the last 6 hours. Another class of acute stroke patients are those who have suffered stroke within the last 4 hours. Another class of acute stroke patients are those who have suffered stroke within the last 3 hours.

According to the method of the invention, treatment of acute stroke, i.e. treatment during the cerebral event causing stroke and the 7 days thereafter, may involve treatment of a selective HDAC8 inhibitor in combination with thrombolytics such as recombinant tissue plasminogen activator (rtPA).

During acute ischemic stroke, the arterial occlusion caused by the thrombus or embolus results in an immediate infarcted core of brain tissue, where cerebral blood flow is significantly reduced, for example to less than 20% of the normal blood flow. The infarcted core suffers irreversible damage due to significant cell death. The length of time that ischemia persists, and the severity of the ischemia, contribute to the extent of the infarct. An area around the infracted core, known as the ischemic penumbra, suffers a delayed and less severe infarct. For example, during acute stroke the penumbra may have a reduction in blood flow of from about 20-40%.

Patients who have suffered stroke more than 24 hours previously often develop cerebral edema which typically occurs at one to five days after stroke. As used herein, the term “cerebral edema” refers to fluid collecting in brain tissue due to cellular swelling and the breakdown of the blood-brain barrier.

A third class of stroke patients to which the selective HDAC8 inhibitors of the present invention may be administered are patients who have suffered stroke more than 7 days previously, who are typically in need of restorative treatment (including enhancing plasticity).

The terms “administration of” or “administering a” compound or a selective HDAC8 inhibitor should be understood to mean providing a compound of the invention to the individual in need of treatment in a form that can be introduced into that individual's body in a therapeutically useful form and therapeutically useful amount.

The terms “effective amount” or “therapeutically effective amount” means the amount of the subject compound or the selective HDAC8 inhibitor that will elicit the biological or medical response of a tissue, system, animal or human that is being sought by the researcher, veterinarian, medical doctor or other clinician.

By “pharmaceutically acceptable” it is meant the carrier, diluent or excipient must be compatible with the other ingredients of the formulation and not deleterious to the recipient thereof.

The term “pharmaceutically acceptable salts” refers to salts prepared from pharmaceutically acceptable non-toxic bases or acids including inorganic or organic bases and inorganic or organic acids. Salts derived from inorganic bases ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins. When the compound of the present invention is basic, salts may be prepared from pharmaceutically acceptable non-toxic acids, including inorganic and organic acids. Such acids include acetic, benzenesulfonic, benzoic, citric, fumaric, gluconic, glutamic, hydrobromic, hydrochloric, lactic, maleic, methanesulfonic, nitric, pamoic, phosphoric, succinic, sulfuric, tartaric, p-toluenesulfonic, trifluoroacetic acid and the like.

The selective HDAC8 inhibitors of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The selective HDAC8 inhibitors of the invention may also be delivered by the use of monoclonal antibodies as individual carriers to which the compound molecules are coupled. The compounds may also be coupled with soluble polymers as targetable drug carriers.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

The pharmaceutical compositions of the present invention encompass any composition made by admixing a compound of the present invention and a pharmaceutically acceptable carrier. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, or pharmaceutically acceptable salts thereof, may also be administered by controlled release means and/or delivery devices.

Pharmaceutical compositions intended for oral use may be prepared according to any method known to the art for the manufacture of pharmaceutical compositions, and may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

Pharmaceutical compositions of the invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of the invention can also be in a form suitable for rectal administration wherein the carrier is a solid. It is preferable that the mixture forms unit dose suppositories. Suitable carriers include cocoa butter and other materials commonly used in the art.

The invention is also directed to a therapeutically effective intravenous formulation of the compounds of the invention, which is solution stable and isotonic with human blood. The intravenous formulation preferably can be packaged in plastic or glass, and meets government and compendial (USP in the US) particulate standards, and can be used as effective therapy to treat stroke.

Pharmaceutical intravenous formulations of the invention will generally include a therapeutically effective amount of a compound of the invention to treat a neurodegenerative disease, in addition to one or more pharmaceutically acceptable excipients. The compositions are advantageously prepared together with liquid inert carriers, such as water. Suitable liquid excipients/carriers are water for injection (US Pharmocoepia) and saline solution. The solution should be pyrogen-free; and also should be absent of particulate matter. Limits for the amount of particulate matter (i.e., extraneous, mobile undissolved substances, other than gas bubbles) which may be found in IV fluids are defined in the US Pharmacoepia.

Other suitable excipients and other additives for intravenous formulations include solvents such as ethanol, glycerol, propylene glycol, and mixtures thereof; stabilizers such as EDTA (ethylene diamine tetraacetic acid), citric acid, and mixtures thereof; antimicrobial preservatives, such as benzyl alcohol, methyl paraben, propyl paraben, and mixtures thereof; buffering agents, such as citric acid/sodium citrate, potassium hydrogen tartrate, sodium hydrogen tartrate, acetic acid/sodium acetate, maleic acid/sodium maleate, sodium hydrogen phthalate, phosphoric acid/potassium dihydrogen phosphate, phosphoric acid/disodium hydrogen phosphate, and mixtures thereof; tonicity modifiers, such as sodium chloride, mannitol, dextrose, and mixtures thereof; fluid and nutrient replenishers such as synthetic amino acids, dextrose, sodium chloride, sodium lactate, Ringer's solution, and other electrolyte solutions.

As used herein, the term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk of onset of atherosclerosis, or atherosclerosis medical condition or atherosclerosis disease event.

An effective amount of a selective HDAC8 inhibitor in the method of this invention is in the range of about 0.001 mg/kg to about 20 mg/kg of body weight per day, preferably 0.01 mg to about 10 mg per kg, and most preferably 0.1 to 1 mg per kg, in single or divided doses. A single daily dose is preferred but not necessary. On the other hand, it may be necessary to use dosages outside these limits in some cases. As examples, the daily dosage amount may be selected from, but not limited to, 25 mg, 50 mg, 75 mg, 100 mg, 125 mg, 150 mg, 200 mg and 250 mg. It will be understood, however, that the specific dose level for any particular patient will depend upon a variety of factors including the age, body weight, general health, sex, diet, time of administration, route of administration, rate of excretion, drug combination and the severity of the patient's condition. A consideration of these factors is well within the purview of the ordinarily skilled clinician for the purpose of determining the therapeutically effective or prophylactically effective dosage amount needed to prevent, counter, or arrest the progress of the condition.

Specific dosages of the compounds of the present invention for oral use, or pharmaceutically acceptable salts thereof, for administration include 1 mg, 5 mg, 10 mg, 30 mg, 80 mg, 100 mg, 150 mg, 300 mg and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions useful for treatment may comprise about 1 mg, 5 mg, 10 mg, 30 mg, 80 mg, 100 mg, 150 mg, 300 mg and 500 mg of active ingredient

In the method of treatment of this invention, the HDAC8 inhibitor may be administered via any suitable route of administration such as orally, parenterally, or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. Oral formulations are preferred.

The compounds of the present invention may be used in combination with one or more other drugs in the treatment of diseases or conditions for which the compounds of the present invention have utility, where the combination of the drugs together are safer or more effective than either drug alone. Additionally, the compounds of the present invention may be used in combination with one or more other drugs that treat, prevent, control, ameliorate, or reduce the risk of side effects or toxicity of the compounds of the present invention. Such other drugs may be administered, by a route and in an amount commonly used therefor, contemporaneously or sequentially with the compounds of the present invention. Accordingly, the pharmaceutical compositions of the present invention include those that contain one or more other active ingredients, in addition to the compounds of the present invention. The combinations may be administered as part of a unit dosage form combination product, or as a kit or treatment protocol wherein one or more additional drugs are administered in separate dosage forms as part of a treatment regimen.

Examples of combinations of the compounds of the present invention include combinations with another stroke agent, for example a COX-2 inhibitor, a nitric oxide synthase inhibitor, a Rho kinase inhibitor, an angiotension II type-1 receptor antagonist, a glycogen synthase kinase 3 inhibitor, a sodium or calcium channel blocker, a p38 MAP kinase inhibitor, a thromboxane AX-synthetase inhibitor, a statin (an HMG CoA reductase inhibitor), a neuroprotectant, a beta andrenergic blocker, a NMDA receptor antagonist, a platelet fibrinogen receptor antagonist, a thrombin inhibitor, an antihypertensive agent, a vasodilator, or a compound which is known to be neuropharmacologically active or is known or believed to be effective in treating stroke.

Exemplary compounds useful in combinations with the compounds of the present invention include acebutolol, altiplase, argatroban, arundic acid, aspirin, atenolol, atorvastatin, candesartan, celecoxib, citicoline, clopidrogel, crobenetine, cyclandelate, dazoxiben, dexanabinol, disufenton, edaravone, enalapril, eprosartan, esmolol, fasudil, fluvastatin, gavestinel, irbesartan, isbogrel, labetalol, lamifiban, lithium ion, losartan, meloxicam, memantine, metoprolol, minocycline, nadolol, nisvastatin, ozagrel, piclozotan, pindolol, pravastatin, propranolol, ramipril, repinotan, ridogrel, rofecoxib, rosuvastatin, simvastatin, sodium 4-phenyl butyrate, telmisartan, ticlopidine, timolol, tirofiban, traxoprodil, Trolox™, uridine, valdecoxib, valproic acid, valsartan, vitamin C, vitamin E, warfarin, SKB 239063, 1400W94, AMG 517, 782443, 705498, V 14380, A425619, PAC20030, BXT-51072, SUNN8075 and SUNN4057, and pharmaceutically acceptable salts thereof.

The following examples are provided so that the invention might be more fully understood. These examples are illustrative only and should not be construed as limiting the invention in any way.

Compounds may be assessed for their inhibition of HDAC subtypes 1, 3 and 8 as follows.

### HDAC1 Assay

The HDAC1 assay is used to quantify the histone deacetylase (HDAC) inhibitory activity of compounds against an affinity purified, recombinant human HDAC1 enzyme. The assay is performed in 96 well microtiter plates by pre-incubating serial dilutions of compounds with a fixed concentration of HDAC1 enzyme complex, and then adding an acetylated lysine-containing substrate/developer that fluoresces upon deacetylation. The deacetylase reaction is performed at 37° C. for 60 min, terminated by addition of the developer solution, and then fluorescence (ex 360 nM, em 460 nM) is measured using a plate reader.

The stock reagents include HDAC1 enzyme, which may be prepared by overexpression of C-terminally tagged (Flag epitope) human HDAC1 in mammalian cells, and an HDAC substrate buffer system. The substrate buffer system include reagents of the HDAC Fluorescent Activity Assay purchased from BioMol Research Laboratories (Plymouth Meeting, Pa.), using the Fluor-de-Lys™ Substrate/Developer System. The reagents include the fluorescent substrate as a 50mM stock solution, and the Developer Concentrate. Deacetylation of the lysine residue of the Fluor-de-Lys substrate is quantified by measuring the fluorescence (ex 360 nM, em 460 nM) after addition of the developer. TSA is provided as a 10 mM stock solution in 100% DMSO.

The working reagents include an assay buffer (25 mM Tris/HCl pH8, 137 mM NaCl, 2.7 mM KCl, 1 mM MgCl₂, 0.1 mg/ml BSA) and a diluted substrate solution. A suitable substrate solution is prepared by diluting the commercial 50 mM Fluor-de-Lys substrate to 150 μM with HDAC assay buffer prior to each use. The final concentration in the assay is 20 μM.

The commercial 20× Developer Concentrate is diluted 1:167 into HDAC Assay Buffer. 2 μM TSA to this solution increased its ability to stop the reaction.

The HDAC1 enzyme is diluted in assay buffer prior to each use from a fresh aliquot of enzyme.

The final concentration in the assay is 1-2 nM.

Test compounds may be prepared as a 10×5% DMSO solution in assay buffer. The final DMSO concentration in the reaction is 0.5%.

The reaction is performed in 96-well microplate in a final volume of 50 ul/well, according to the following steps:

1) Add 5 ul of DMSO/compound solution

2) Add 35 ul of HDAC1 enzyme in assay buffer (or 35 ul assay buffer in the negative controls)

3) Incubate 10 min at rt

4) Start the reaction by adding 10 ul of the 150 μM Substrate Solution

5) Incubate 1 h at 37° C.

6) Stop by adding 50 ul of Developer/4 μM TSA solution

7) Incubate 10 min at rt

8) Measure the fluorescence at Ex.360 nM and Em.460 nM

The negative controls are performed with the substrate alone, without HDAC1 enzyme.

### HDAC3 Assays

The HDAC3 assay is used to quantify the HDAC inhibitory activity of compounds against an affinity purified, recombinant human HDAC3 enzyme. The assay is performed in 96 well microtiter plates by pre-incubating serial dilutions of compounds with a fixed concentration of HDAC3 enzyme complex and then adding an acetylated lysine-containing substrate/developer that fluoresces upon deacetylation. The deacetylase reaction is performed at 37° C. for 60 min, terminated by addition of the developer solution, and then fluorescence (ex 360 nM, em 460 nM) is measured using a plate reader.

The stock reagents include HDAC3 enzyme, which may be prepared by overexpression of HDAC3 C-terminally tagged (Flag epitope)/DAD co-activator complex in mammalian cells, and HDAC Substrate Buffer System. The substrate buffer system comprises reagents of the HDAC Fluorescent Activity Assay purchased from BioMol Research Laboratories (Plymouth Meeting, Pa.), and uses the Fluor-de-Lys™ Substrate/Developer System. The reagents include the fluorescent substrate as a 50 mM stock solution, and the Developer Concentrate. Deacetylation of the lysine residue of the Fluor-de-Lys substrate is quantitated by measuring the fluorescence (ex 360 nM, em 460 mM) after addition of the Developer.

TSA is provided as a 10 mM stock solution in 100% DMSO.

The working reagents include an assay buffer (25 mM Tris/HCl pH8, 137 mM NaCl, 2.7 mM KCl, 1 mM MgCl₂, 0.1 mg/ml BSA) and a diluted substrate solution. The commercial 50 mM Fluor-de-Lys substrate is diluted to 150 μM with HDAC assay buffer prior to each use. The final concentration in the assay is 20 μM.

The commercial 20× Developer Concentrate (KI-105) is diluted 1:167 into HDAC Assay Buffer. 2 μM TSA added to this solution increases its ability to stop the reaction.

The HDAC3(DAD) enzyme complex is diluted in assay buffer prior to each use from a fresh aliquot of enzyme. The final concentration in the assay is 1-2 nM.

Test compounds may be prepared as a 10×5% DMSO solution in assay buffer. The final DMSO concentration in the reaction is 0.5%.

The reaction is performed in 96-well microplate in a final volume of 50 ul/well, according to the following steps:
1) Add 5 ul of DMSO/compound solution
2) Add 35 ul of HDAC3 enzyme in assay buffer (or 35 ul assay buffer in the negative controls)
3) Incubate 10 min at rt
4) Start the reaction by adding 10 ul of the 150 μM Substrate Solution
5) Incubate 1 h at 37° C.
6) Stop by adding 50 ul of Developer/4 μM TSA solution
7) Incubate 10 min at rt
8) Measure the fluorescence at Ex.360 nM and Em.460 nM
   The negative controls are performed with the substrate alone, without enzyme.

### HDAC8 Assay

In this assay an E. coli expressed human HDAC8-C-His C-terminal-full length (starting from amino acid 1 to 377+ linker IEGRGS+6 His) is used. The HDAC8 assay is used to measure inhibitory activity of compounds against the human HDAC8 enzyme. The assay may be performed in 96 well or 384 well microtiter plates by pre-incubating serial dilutions of compounds with a fixed concentration of HDAC8 enzyme and then adding a diacetylated peptide-containing substrate/developer that fluoresces upon deacetylation. The deacetylase reaction is performed at 37° C. for 60 min and stopped by addition of the developer solution. Thereafter fluorescence (ex 360 nM, em 460 nM) is measured.

HDAC 8-C is transformed into E. coli BL21 codon plus cells and protein expression is induced upon addition of IPTG. After overnight incubation at 18° C., cells are lysed in an appropriate buffer and the HDAC 8-C protein is purified in three steps, a Nickel-Chelating affinity chromatography, an anion-exchange (MonoQ) chromatography and a size exclusion chromatography on column G75(gel filtration).

Reagents of the HDAC Fluorescent Activity Assay are purchased from BioMol Research Laboratories (Plymouth Meeting, Pa.), and include the Fluor-de-Lys™-HDAC8 Substrate/DeveloperII System. The reagents include the fluorescent substrate as a 5 mM stock solution, the and the DeveloperII Concentrate. Deacetylation of the diacetylated peptide residue of the Fluor-de-Lys substrate is quantitated by measuring the fluorescence (ex 360 nM, em 460 nM) after addition of Developer II.

TSA is provided as a 10 mM stock solution in 100% DMSO.

The working reagents include an assay buffer (20 mM Hepes pH 8, 137 mM NaCl, 2.7 mM KCl, 1 mM MgCl₂, 0.1 mg/ml BSA, 0.2% N-Octyl Glucoside), and a diluted substrate solution. The commercial 5 mM Fluor-de-Lys substrate (KI-178) is diluted to 1 mM with distilled water prior to each use. The final concentration in the assay is 70 μM.

The commercial 5× Developer Concentrate (KI-176) is diluted at 1× into distilled water. 10 μM LAQ to this solution increases its ability to stop the reaction.

The enzyme is diluted in 1.25× assay buffer prior to each use from a fresh aliquot of enzyme.

The final concentration in the assay is 10-15 nM.

Test compounds are prepared as a 10×5% DMSO solution in distilled water. The final DMSO concentration in the reaction is 0.5%.

The experiment is performed in a 96-well microplate (Matrix Screenmates white), 50 ul volume, according to the following steps:

1) Add 5 ul of DMSO/compound solution

2) Add 40 ul of HDAC8 enzyme in assay buffer

3) Incubate 15 min at rt

4) Start the reaction by adding 5 ul of the 1 mM Substrate Solution

5) Incubate 1 h at 37° C.

6) Stop by adding 50 ul of Developer II/10 μM LAQ solution

7) Incubate 30 min at rt

8) Measure the fluorescence at Ex.360 nM and Em.460 nM

The experiment is performed in a 384-well microplate (Matrix Screenmates white), 25 ul volume, according to the following steps:

1) Add 2.5 ul of DMSO/compound solution

2) Add 20 ul of HDAC8 enzyme in assay buffer

3) Incubate 15′ at rt

4) Start the reaction by adding 2.5 ul of the 1 mM Substrate Solution

5) Incubate 1 h at 37° C.

6) Stop by adding 25 ul of Developer/10 μM LAQ solution

7) Incubate 30 min at rt

8) Measure the fluorescence at Ex.360 nM and Em.460 nM

The following pharmaceutically active compounds, all of which are taught in the prior art, were assessed for the inhibition of various HDAC subtypes the preceding cell-free, isoform selective HDAC enzyme assays:

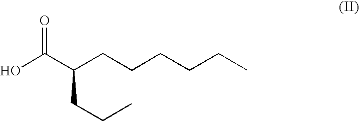

(2R)-2-propyloctanoic acid (arundic acid);

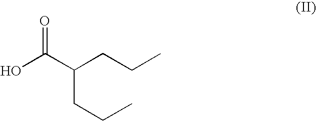

2-propylpentanoic acid (valproic acid);

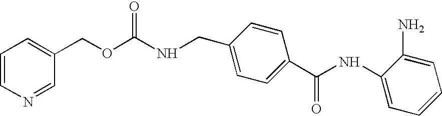

N-(2-Aminophenyl)-4-[N-(pyridine-3-yl-methoxycarbonyl)aminomethyl] benzamide (MS 27-275) (see Joboin et al, Cancer Res. 2002, 62, 6108-6115); and

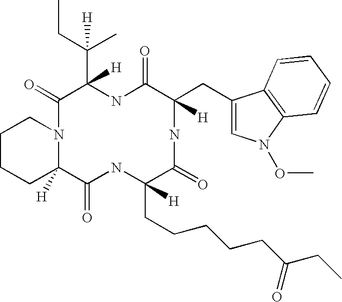

apicidin.

The data is presented below in Table 1:

TABLE 1
IC₅₀ Inhibition (μM) of Selected Compounds for HDAC
Isoforms 1, 3, 8 and 11

| | HDAC | Arundic | Valproic | | |
|---|---|---|---|---|---|
| HDAC Class | Isoform | acid | acid | Apicidine | MS 27-275 |
| I | HDAC1 | 10 | 50 | 0.020 | 0.20 |
| | HDAC3 | 36 | 40 | 0.014 | 0.73 |
| | HDAC8 | 31 | >50 | >5 | >5 |
| IIa | HDAC4 | >50 | >25 | >5 | 2.5 |
| IIb | HDAC6 | >50 | >5,000 | >0.2 | >5 |
| III | SIRT1 | >50 | | | |

The assay results demonstrate that (2R)-2-propyloctanoic acid (arundic acid) inhibits HDAC with micromolar potency. The assay has further shown that arundic acid inhibits HDAC 8 with a potency similar (within 5-fold) to HDAC1 and HDAC3. The profile of arundic acid differs from the profile of other HDAC inhibitors, such as N-hydroxy-4-{methyl[(5-pyridin-2-yl-2-thienyl)sulfonyl]amino}benzamide, trichlorostatin A (“TSA”), suberoyl anilide hydroxamic acid (“SAHA”), MS-275, and apicidin, all of which are more than 25 fold selective for HDAC1 and HDAC3 over HDAC 8. See Vannini, PNAS101, 42:15065:

The following abbreviations are used throughout the text:

TSA: trichlorostatin A

BSA: bovine serum albumin

DMSO: dimethyl sulfoxide

rt: room temperature

h: hours

min: minutes

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method of treating a neurodegenerative disease, comprising administering a therapeutically effective amount of a selective HDAC8 inhibitor to a patient in need of such treatment, wherein the selective HDAC8 inhibitor is not (2R)-2-propyloctanoic acid.

2. The method of claim 1 wherein the HDAC 8 inhibitor is a selective HDAC8 inhibitor which is selective for each of the HDAC1, HDAC3 and HCDAC8 subtypes within a factor of five.

3. The method of claim 1, wherein the HDAC8 inhibitor has an IC₅₀ for each of HDAC1, HDAC3 and HDAC8 of less than of less than or equal to 100 PM.

4. The method of claim 3, wherein the HDAC8 inhibitor has an IC₅₀ for each of HDAC1, HDAC3 and HDAC8 of less than or equal to 50 μM.

5. The method of claim 4, wherein the HDAC8 inhibitor has an IC₅₀ for each of HDAC1, HDAC3 and HDAC8 of less than or equal to 25 μM.

6. The method of claim 5, wherein the HDAC8 inhibitor has an IC₅₀ for each of HDAC1, HDAC3 and HDAC8 of less than or equal to 1000 nM.

7. The method of claim 6, wherein the HDAC8 inhibitor has an IC₅₀ for each of HDAC1, HDAC3 and HDAC8 of less than or equal to 500 nM.

8. The method of claim 7, wherein the HDAC8 inhibitor has an IC₅₀ for each of HDAC1, HDAC3 and HDAC8 of less than or equal to 250 nM.

9. The method of claim 8, wherein the HDAC8 inhibitor has an IC₅₀ for each of HDAC1, HDAC3 and HDAC8 of less than or equal to 100 nM.

10. The method of claim 9, wherein the HDAC8 inhibitor has an IC₅₀ for each of HDAC1, HDAC3 and HDAC8 of less than or equal to 50 nM.

11. The method of claim 1, wherein the neurodegenerative disease is selected from the group consisting of vascular dementia, pre-senile and senile dementia, Alzheimer's Disease, multiple sclerosis, amyotrophic lateral sclerosis, Creutzfeldt-Jakob Disease, stroke, traumatic brain injury, spinal cord injury, Pick's disease, Huntington's Disease, progressive supranuclear palsy, Lewy body disease, corticobasal degeneration, Parkinson's Disease, Tourette's Syndrome, familial tremor, torsion dystonia, cerebellar degenerations, spinocerebellar degeneration, Charcot-Marie-Tooth disease, chronic progressive neuropathies, retinitis pigmentosa and hereditary optic atrophy.

12. The method of claim 11, wherein the neurodegenerative disease is selected from the group consisting of Alzheimer's Disease, multiple sclerosis, amyotrophic lateral sclerosis, Creutzfeldt-Jakob Disease, stroke, traumatic brain injury, spinal cord injury, Pick's disease, Huntington's Disease, progressive supranuclear palsy, Lewy body disease and Parkinson's Disease.

13. The method of claim 12, wherein the neurodegenerative disease is stroke.

14. The method of claim 13, wherein the neurodegenerative disease is ischemic stroke.

15. A method for identifying one or more compounds useful for treatment of neurodegenerative diseases from a group of compounds, comprising the steps of
(a) assaying the binding affinity of a group of compounds for each of the HDAC1, HDAC3 and HDAC8 subtypes,
(b) determining the IC₅₀ value for each of the compounds in the group for each of the HDAC1, HDAC3 and HDAC8 subtypes, and
(c) selecting one or more compounds from the group in which the IC₅₀ value for each of the HDAC1, HDAC3 and HDAC8 subtypes is within a factor of five.

16. The method of claim 11, further comprising (d) selecting one or more compounds having an IC₅₀ value for HDAC8 of less than a threshold value.

17. The method of claim 12, wherein the threshold value is selected from the group consisting of 100 μM, 50 μM, 25 μM, 10 μM, 1000 nM, 500 nM, 250 nM, 100 nM and 50 nM.

18. (canceled)
